(19) United States
(12) Patent Application Publication
(10) Pub. No.: US 2023/0077337 A1
(43) Pub. Date: Mar. 16, 2023

(54) MAGNETICALLY STEERABLE SCREW-TIP CANNULA

(71) Applicant: University of Utah Research Foundation, Salt Lake City, UT (US)

(72) Inventors: Alan D. Kuntz, Salt Lake City, UT (US); Jacob J. Abbott, Salt Lake City, UT (US); Trevor J. Schwehr, Salt Lake City, UT (US)

(21) Appl. No.: 17/943,622

(22) Filed: Sep. 13, 2022

Related U.S. Application Data
(60) Provisional application No. 63/243,465, filed on Sep. 13, 2021.

(51) Int. Cl. A61M 25/01

(52) U.S. Cl. CPC A61M 25/0127

(57) ABSTRACT

A magnetically steerable screw-tip cannula can include a flexible cannula with a distal end to be inserted into biological tissue and an internal lumen extending to the distal end. A magnetically steerable screw-tip can be on the distal end of the cannula. The screw-tip can include a screw body with screw threads on an exterior surface thereof, the screw threads oriented to generate insertion or retraction force along a longitudinal axis of the screw body when the screw body rotates. The screw body can also include an internal lumen extending along the longitudinal axis of the screw body, where the internal lumen is connected to the internal lumen of the cannula. A magnet can be associated with the screw body having an average magnetization substantially parallel to the longitudinal axis of the screw body.

### RELATED APPLICATION

This application claims priority to U.S. Provisional Application No. 63/243,465, filed Sep. 13, 2021, which is incorporated herein by reference.

### BACKGROUND

Targeted biopsy and therapy in the brain, often achieved using straight cannula insertion, is one approach to the diagnosis and treatment of multiple diseases including brain cancer, epilepsy, and Parkinson’s disease. By inserting cannulas deep in the brain, diagnosis via biopsy and therapies such as gene therapy compounds, cell therapy, cellucidal compounds, and laser and radiofrequency ablation can be delivered directly to the site in the brain at which they can be the most effective. However, there is significant risk associated with the insertion of traditional, straight cannulas into the brain during such procedures. Potential complications include intraparenchymal hemorrhages, leading to paralysis, aphasia, blindness, and other serious neurological problems. In the case of stereotactic brain biopsy, serious complications occur in more than 7% of patients, with symptomatic hemorrhage occurring in more than 4%, and a mortality rate between 1.3-3.7%. Further, non-diagnostic yield for this procedure is reported to be as high as 13%. The location of the lesion greatly affects these concerns as well, with the risk of non-diagnostic yield more than three times higher for lesions located in the deep brain than for those in superficial locations, and the risk of hemorrhage increasing more than four times for deep brain lesions.

To minimize the risk of complications, surgeons can choose safer trajectories, favoring entry points in non-eloquent areas of the brain and paths that avoid sulci, blood vessels, and ventricles. However, these limits constrain the targets surgeons can safely approach. The use of lengthy, linear trajectories that traverse normal, uninvolved brain tissue, can expose the normal brain tissue to excessive force and resulting risk of hemorrhage or other damage. This makes it particularly difficult to reach sites deep in the brain for which a safe, straight-line path to the site does not exist. Further, frequently the delivery of targeted therapy involves delivery of the therapy at multiple sites along the path of the cannula where the goal is to closely match the volume of the pathological site, e.g., shape of the tumor, while minimizing therapy delivery to healthy tissue. This results in the construction of a “snowman” type volume created from the union of therapy volumes deployed at multiple sites. In the case of many tumor volumes, it can be difficult to form a volume that matches the shape of the tumor when using a straight-line cannula access to the tumor.

### SUMMARY

In one example, a magnetically steerable screw-tip cannula can include a flexible cannula that includes a distal end to be inserted into biological tissue. The cannula can have an internal lumen extending to the distal end. A magnetically steerable screw-tip can be on the distal end of the cannula. The screw-tip can include a screw body that includes screw threads on an exterior surface thereof. The screw threads can be oriented to generate insertion or retraction force along a longitudinal axis of the screw body when the screw body rotates. The screw body can also include an internal lumen extending along the longitudinal axis of the screw body. The internal lumen can be connected to the internal lumen of the cannula. The screw-tip can also include a magnet attached to the screw body. The magnet can have an average magnetization substantially parallel to the longitudinal axis of the screw body.

In another example, a system can include a magnetically steerable screw-tip cannula as described above. The system can also include a torque applying mechanism at a position toward a proximal end of the flexible cannula. The torque applying mechanism can be configured to apply torque to the flexible cannula so that the flexible cannula transfers the torque to the magnetically steerable screw-tip to generate insertion or retraction force.

In yet another example, a method of using a magnetically steerable screw-tip cannula can include inserting a magnetically steerable screw-tip cannula into biological tissue. The magnetically steerable screw-tip cannula can include a flexible cannula having a distal end that is inserted into the biological tissue and an internal lumen extending to the distal end. A magnetically steerable screw-tip can be on the distal end of the cannula. The screw-tip can include a screw body having screw threads on an exterior surface thereof and an internal lumen extending along a longitudinal axis of the screw body. The internal lumen can be connected to the internal lumen of the cannula. The screw-tip can also include a magnet attached to the screw body. Mechanical torque can be applied to the flexible cannula at a location outside the biological tissue. The cannula can transfer the torque to the screw-tip so that the screw-tip rotates and the screw threads generate insertion or retraction force along the longitudinal axis of the screw body. A magnetic field can be applied from a magnetic field source positioned outside the biological tissue. The magnetic field can impinge upon the magnet of the magnetically steerable screw-tip cannula to generate a force or torque causing a deformation of the cannula to steer the cannula.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1A is a side view of an example magnetically steerable screw-tip cannula in accordance with the present disclosure.

FIG. 1B is a side cross-sectional view of the example magnetically steerable screw-tip cannula of FIG. 1A.

FIG. 2 is a side view of another example magnetically steerable screw-tip cannula in accordance with the present disclosure.

FIG. 3 is a side view of yet another example magnetically steerable screw-tip cannula in accordance with the present disclosure.

FIG. 4 is a schematic view of an example system including a magnetically steerable screw-tip cannula being inserted into biological tissue, in accordance with the present disclosure.

FIG. 5 is a flowchart illustrating an example method of using a magnetically steerable screw-tip cannula in accordance with the present disclosure.

FIG. 6 is a cross-sectional view of an example magnetically steerable screw-tip cannula being used to treat biological tissue in accordance with the present disclosure.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “a channel” includes reference to one or more of such channels and reference to “the magnet” refers to one or more of such magnets.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term "at least one of' is intended to be synonymous with "one or more of." For example, "at least one of A, B and C" explicitly includes only A, only B, only C, or combinations of each.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### Example Embodiments

As explained above, it can be difficult to safely reach certain portions of brain tissue using a straight cannula. Steerable cannulas have been developed as one way to address this issue. Steerable cannulas can use curvilinear paths in the body to enable access to anatomical targets that are not safely accessible by straight cannulas. Steerable cannulas, of a variety of designs, have received a great deal of attention for potential therapeutic applications in soft tissue such as the liver, lungs, kidneys, prostate, and brain. Steerable cannulas have the potential to decrease the risk of access (and subsequent biopsy or therapy delivery) deep in the brain by enabling steering around sensitive structures such as vasculature. Their ability to curve in tissue also enables honing in on the target during deployment, potentially improving diagnostic yield. Further, as they can curve in tissue, steerable cannulas may provide the ability to more closely match the volume of non-linearly shaped anatomical features, such as tumors, during multi-step therapy delivery.

Existing steerable cannula designs are insufficient for deployment in the brain, however. Many steerable-cannula designs are introduced into the body by pushing the cannulas at their proximal end into the tissue (as with traditional straight instruments). The cannulas can curve in the tissue, but pushing a curved cannula from its base can introduce significant forces on the surrounding tissue at the areas of curvature. In particular, the force applied to the base, or proximal end, of the cannula in order to insert the cannula can cause the curved portion of the cannula to push against surrounding tissue. These forces can result in damage to tissue via contusion or bleeding due to compression and can even result in events in which the tissue around the cannula fractures and the cannula shaft cuts laterally through the tissue causing significant damage.

Further, because of the shaft stiffness of these types of cannulas, which allows them to be pushed from their base through the tissue, the cannulas may not be capable of achieving tight radii of curvature. A recent state-of-the-art steerable-cannula design, specifically tailored to safely achieve tight steering curvature, produces a radius of curvature of approximately 60 mm in ex vivo ovine brain and gelatin tissue phantom. Another notable, biologically inspired steerable cannula has been shown to achieve curvatures of -50 mm, but at the cost of a larger overall diameter of 2.5 mm with significantly smaller working channels due to its segmented design. These achievable curvatures are still insufficient for dexterous navigation and therapy delivery in the brain.

Another paradigm in steerable cannulas can be actuated via internal mechanisms. Examples include tendon-actuated cannulas and concentric tube robots. To deploy deep in sensitive tissue, these robots can employ a ‘follow-the-leader’ motion, in which the robot’s shaft does not change shape after it has been embedded in the tissue. This is possible for these designs, but to do so severely limits the design and prevents them from sufficiently correcting their motion during deployment.

This disclosure describes a new type of steerable cannula that can provide better safety and flexibility compared to previous straight and steerable cannulas. The steerable cannulas described herein can be steered magnetically, and insertion force can be provided by a screw-tip so that the cannula is pulled from the tip instead of pushed from outside the biological tissue. The design can include a screw with a central lumen, which serves as the tip of the cannula. One or more magnets, such as axially magnetized ring magnets (e.g. NdFeB or comparable magnetic material) can be attached to the screw, and a flexible cannula tube can be towed behind the screw and magnets. The cannula can be steered via a robotically controlled external magnet (e.g., a spherical-actuator-magnet manipulator (SAMM) system), a manually controlled external magnet (e.g., using an image guided-surgery method, such as the TRAC method), or a set of stationary external electromagnets (e.g., an Omnimagnet, an OctoMag system, or similar). In some examples, the magnetic field can be generated by an omnidirectional magnet described in U.S. Pat. No. 10,199,147, which is incorporated herein by reference. A robotic insertion/rotation stage at the proximal end of the tube (outside of the tissue) can rotate the tube (and thus the screw). The insertion/rotation stage can also advance the tube in a coordinated fashion, based on the pitch of the screw, to keep up with the pulling from the tip rather than pushing the tip forward. Alternatively, the rotation and/or insertion actions of this robotic insertion/rotation stage can be performed manually, rather than robotically, even as steering is driven via the external magnetic field.

The flexible cannula being pulled by the screw-tip can serve as a working channel after it is steered to the desired site in the brain for, e.g., biopsy tools, targeted liquid drugs, or ablation probes. Motion planning and image-based closed-loop control can be used to steer the cannula to a desired site.

The screw-tip flexible cannulas described herein can achieve smaller radii of curvature than many existing devices while exerting less force on brain tissue during deployment. This can enable safer and more accurate access to sites deeper in the brain due to the ability to steer around sensitive anatomical structures, hone in on targets, and match anatomical volumes with multi-stage therapy delivery. The screw-tip flexible cannulas can also be used in other organs where similar procedures may be useful, such as the liver and the lungs.

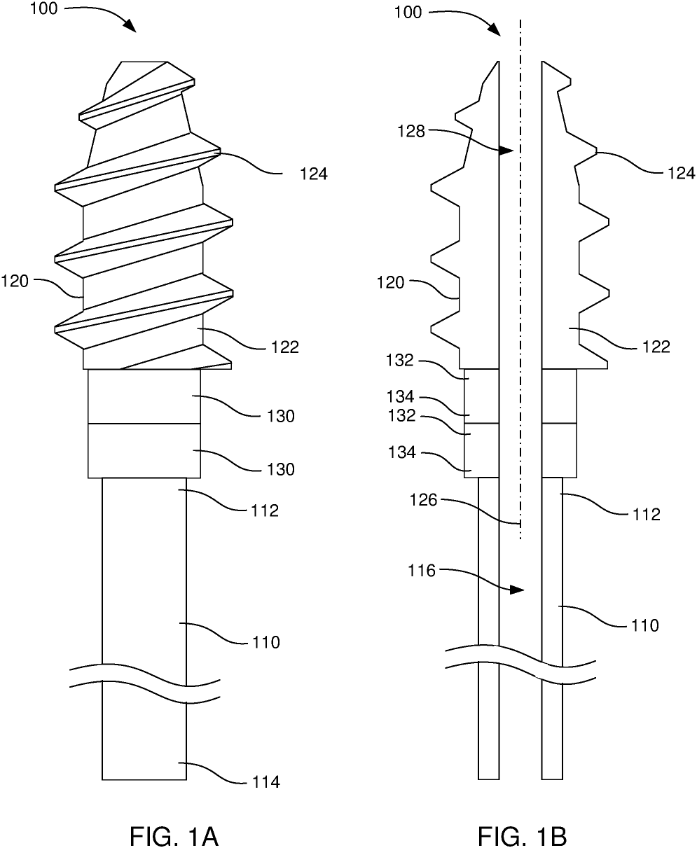

With this general description in mind, FIG. 1A shows a side view of an example magnetically steerable screw-tip cannula 100. This device includes a flexible cannula 110 with a distal end 112 and a proximal end 114 opposite from the distal end. As used herein, the “distal” end of the cannula is the end that is inserted into biological tissue, such as a brain. The proximal end is the opposite end, which remains outside the biological tissue and outside the patient in most cases. FIG. 1B is a cross-sectional view of the same magnetically steerable screw-tip cannula. This figure shows an internal lumen 116 extending to the distal end 112 of the cannula. The device also includes a magnetically steerable screw-tip 120 on the distal end of the cannula. The screw-tip includes a screw body 122 with screw threads 124 on an exterior surface of the screw body. The screw threads have a helical shape and are oriented to generate insertion or retraction force along a longitudinal axis 126 of the screw bod when the screw body rotates. The screw body also includes an internal lumen 128 extending along the longitudinal axis of the screw body. This internal lumen is connected to the internal lumen of the flexible cannula. The screw-tip also includes magnets 130 attached to the screw body. One magnet or multiples magnets can be used, but this example includes two permanent magnets having a ring shape, attached to the screw body. The magnets have north poles 132 and south poles 134 oriented so that the average magnetization direction (i.e., the vector from the south pole to the north pole) is substantially parallel to the longitudinal axis of the screw body. The ring-shaped magnets also have an internal lumen that is connected to the internal lumen of the flexible cannula and the internal lumen of the screw body. Thus, the internal lumens provide a clear pathway to deliver materials or instruments to a site at the tip of the screw-tip.

The magnet in the magnetically steerable screw-tip can allow forces and torque to be applied to the screw-tip using a magnetic field. For example, a magnetic field can be applied by a magnetic field source located outside the biological tissue, and this magnetic field can exert a force and/or torque on the screw-tip inside the biological tissue. As in the example described above, the magnet can have an average magnetization that is substantially parallel to the longitudinal axis of the screw body. When an external magnetic field is applied, the magnet in the screw-tip can tend to align its magnetic field with the external magnetic field. Therefore, the external magnetic field can be applied in a desired direction to cause the screw-tip of the cannula to move and/or rotate to align with the magnetic field direction. Thus, a force, or a torque, or both can be applied to the screw-tip using the external magnetic field. As a result, the magnetic steering can be invariant with respect to the current rotation of the cannula and screw-tip.

In some examples, the magnet in the screw-tip can be a permanent magnet. In certain cases, the permanent magnet can be a rare-earth magnet. Materials that can be used in permanent magnets include iron, steel, nickel, cobalt, gadolinium, dysprosium, ferrite, alnico, neodymium, samarium, composites thereof, alloys thereof, and the like. In one example, the magnet can be a NdFeB permanent magnet. In other examples, the magnet can be made of a “soft” magnetic material that becomes magnetized in the presence of an applied magnetic field. Examples of soft magnetic materials include alloys of iron, nickel, cobalt, with elements such as boron, carbon, phosphorous, and silicon. As mentioned above, one magnet or multiple magnets can be used. For example, the screw-tip can include two, three, four, six, eight or more magnets. The magnets can be stacked together or placed in separate locations in or on the screw body. In certain examples, the magnets can be stacked at a proximal end (opposite from the tip) of the screw body. In another alternative example, the screw body can be formed of the magnets (i.e. the screw body can be molded or machined from a magnetic material).

The magnet or magnets of the screw-tip can include an internal lumen. The internal lumen of the magnets can be aligned with the internal lumen of the screw body and the internal lumen of the flexible cannula so that a clear pathway exists through the flexible cannula all the way to the tip of the screw-tip. In certain examples, the magnet can be in the shape of a ring or cylinder with an internal lumen in the center of the ring of cylinder. The internal lumen of the magnet can be coaxial with the internal lumen of the screw body and the internal lumen of the flexible cannula in some examples. In further examples, the magnet can be attached to the proximal end of the screw body and the internal lumen of the magnet can be directly connected to the internal lumen of the screw body. The flexible cannula can also be directly connected to the internal lumen of the magnet so that fluid delivery or instrument delivery can be accomplished through the flexible cannula, then through the internal lumen of the magnet, then through the internal lumen of the screw body. The magnet can be connected to the flexible cannula and to the screw body by any suitable attachment method, such as gluing, friction fitting, welding, and so on. In other examples, the flexible cannula can be inserted into or through the internal lumen of the magnet. The flexible cannula can also be inserted into or through the internal lumen of the screw body.

The magnet can have an internal lumen with a diameter from about 0.05 mm to about 5 mm in some examples, or from about 0.08 mm to about 3 mm, or from about 0.1 mm to about 2 mm, or from about 0.5 mm to about 1.5 mm. In overall dimensions, the magnet can have an external diameter from about 0.08 mm to about 8 mm, or from about 0.1 mm to about 5 mm, or from about 0.5 mm to about 3 mm, or from about 1 mm to about 2 mm. These dimensions can be for magnets having a circular shape. Magnets of other shapes can also be used, and may have similar dimensions for their width and length. The thickness of the magnet can be from about 0.05 mm to about 5 mm, or from about 0.1 mm to about 3 mm, or from about 0.5 mm to about 2 mm, in some examples.

Magnets without an internal lumen can also be used. If the screw-tip includes a magnet without an internal lumen, then the magnet can be attached to the screw body at a location that does not block the internal lumen of the screw body or the internal lumen of the flexible cannula. For example, small magnets can be arranged around the lumen of the screw body. These can be attached to the exterior of the screw body, or to the proximal end of the screw body, or embedded into the screw body in various examples. In certain examples, a first magnet can be located nearer to the tip, or distal end, of the screw body, and a second magnet can be located nearer to the proximal end of the screw body. The magnets can be aligned so that their average magnetization is substantially parallel to the longitudinal axis of the screw body.

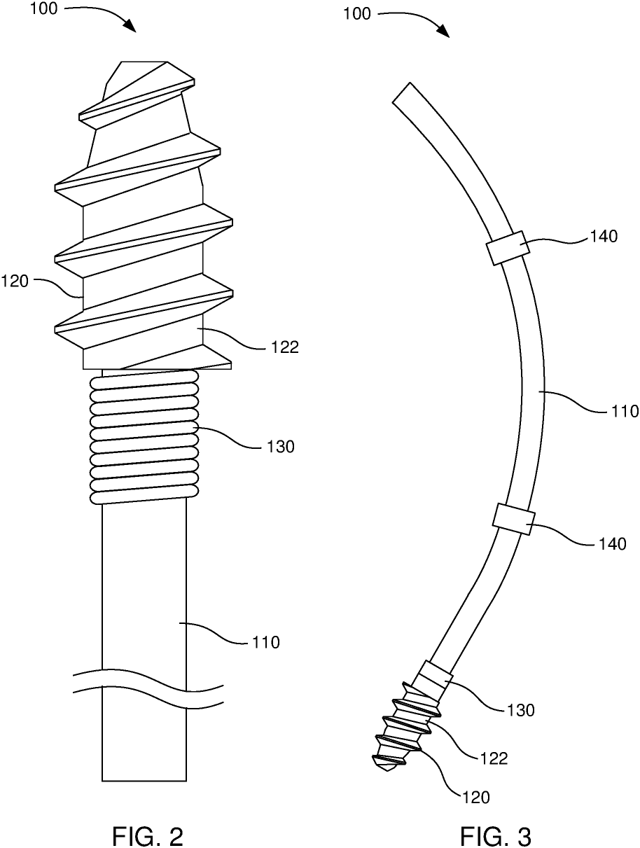

An electromagnet can also be used in the screw-tip instead of or in addition to the types of magnets described above. FIG. 2 shows an example magnetically steerable screw-tip cannula 100 that includes an electromagnet 130. The electromagnet includes a coil of wire wrapped around the flexible cannula 110. In other examples, the coil can be wrapped around the screw body 122 or another component such as a metal core or metal or plastic tube. In any of these examples, the electromagnet can be considered attached to the screw body as a part of the screw-tip 120. The coil can be connected to an electric current source to cause an electric current that generates a magnetic field from the coil. The electric current source can be a power supply that is external to the biological tissue in which the cannula is inserted, and the electromagnet can be connected to the power supply by wires that run along or through the cannula. Alternatively, the electromagnet can be connected to a battery that is in or attached to the screw tip or cannula. In this example, the electromagnet can be run continuously or selectively turned on when it is desired to apply a force or torque to the screw-tip. The current passing through the coil of the electromagnet can also be controlled to adjust the strength of the magnetic field generated by the electromagnet. Using current control requires a simpler model of the system for accurate control than would be required in voltage control. This can be used to adjust the force or torque exerted on the screw tip by an external magnetic field.

Additional magnets can also be located along the length of the flexible cannula. For example, additional ring-shaped magnets can be threaded on or embedded in the cannula with the cannula passing through the interior lumen of the ring-shaped magnets. FIG. 3 shows an example magnetically steerable screw-tip cannula 100 that includes additional magnets 140 along the length of the flexible cannula 110. These additional magnets can provide additional control over the movement of the cannula when the cannula is inserted into biological tissue. For example, the individual magnets can be targeted using an external magnetic field to apply a force and/or torque to the individual magnets. In certain examples, a force can be applied that is parallel to the cannula to assist with insertion or retraction of the cannula. In other examples, a force can be applied that is perpendicular to the cannula to move the cannula in a sideways direction instead of in a forward, insertion direction or a backward, retraction direction. In one example, the additional magnets can be oriented having a polarization opposite to that of the primary magnets in the screw-tip. The example shown in FIG. 3 also includes a screw-tip 120 with a screw body 122 and magnets 130 as in the previous examples. The magnets in the screw-tip and the additional magnets along the length of the flexible cannula can be the same type of magnets or different types of magnets, and may be any of the types of magnets described above.

If magnets are included along the length of the cannula, these additional magnets can have similar dimensions to the magnet dimensions described above. The magnets can be spaced apart along the cannula at a spacing distance from about 5 mm to about 10 cm in some examples, or from about 100 mm to about 5 cm, or from about 1 cm to about 3 cm.

It is noted that the flexible cannulas shown in the drawings are not drawn to scale, and the flexible cannula can often be much longer compared to the length of the screw-tip. In various examples, the flexible cannula can have a length from about 5 cm to about 100 cm, or from about 8 cm to about 80 cm, or from about 10 cm to about 50 cm, or from about 10 cm to about 30 cm. The inner diameter of the cannula can be from about 0.05 mm to about 5 mm in some examples, or from about 0.08 mm to about 3 mm, or from about 0.1 mm to about 2 mm, or from about 0.5 mm to about 1.5 mm. The outer diameter of the cannula can be from about 0.08 mm to about 8 mm, or from about 0.1 mm to about 5 mm, or from about 0.5 mm to about 3 mm, or from about 1 mm to about 2 mm.

The flexible cannula can be formed of any material which is biocompatible and provides sufficient mechanical integrity to transfer torque to the screw-tip while also avoiding or minimizing damage to contacted tissue. Non-limiting examples of suitable materials can include polymer material such as PTFE, TYGON, and the like, metal material, composites, and the like. For example, rigid metal may be segmented and held together via flexible linkages such as polymer links, metal links, or the like. In certain examples, the flexible cannula can be machined to increase its compliance, such as by reducing sidewall thickness, selective patterning (e.g. laser etching patterns), and/or selective removal (e.g. notches). As a general guideline, the flexible cannula can be designed with the goal of achieving high torsional stiffness and low bending stiffness.

The screw body can similarly be formed of a suitable biocompatible material. Generally, the screw body can be formed of a material which is more rigid than the cannula, and in some cases is non-flexible. Non-limiting examples can include brass, stainless steel, titanium, aluminum, Nitinol, and hard plastics such as ABS, PVC, polypropylene, polycarbonate, polystyrene, polyethylene, polyurethane, PET, DELRIN® (available from DuPont, USA), and the like.

The screw body can have an outer diameter that is similar to or slightly larger than the flexible cannula. The outer diameter of the screw body can be defined as the outer diameter at the widest portion of the screw body, not including the screw threads. The screw threads can extend further out from the outer diameter of the screw body. In some examples, the outer diameter of the screw body can be from inner diameter of the cannula can be from about 0.08 mm to about 8 mm, or from about 0.1 mm to about 5 mm, or from about 0.5 mm to about 3 mm, or from about 1 mm to about 2 mm. The screw body can have an internal lumen as explained above. The inner diameter of the screw body can be from about 0.05 mm to about 5 mm in some examples, or from about 0.08 mm to about 3 mm, or from about 0.1 mm to about 2 mm, or from about 0.5 mm to about 1.5 mm.

The screw threads of the screw body can extend outward by a distance from about 0.04 to about 4 mm, or from about 0.08 to about 3 mm, or from about 0.1 to about 2 mm, or from about 0.5 to about 2 mm. The screw threads can have any suitable shape. The examples shown in the figures have screw threads with a truncated triangular cross-section. In other examples, the screw can have a sharp pointed cross-section, or a rounded cross-section, a square cross-section, or other shaped cross-section. The threads can have any suitable pitch, such as from 0.05 mm to 3 mm, or from 0.08 mm to 2.5 mm, or from 0.1 mm to 2 mm, or from 0.5 mm to 1.5 mm. The screw body can also have a tapered distal end or tip to help penetrate into biological tissue.

In addition to the magnetically steerable screw-tip cannulas themselves, the present disclosure also describes systems that include the steerable cannulas and which can be used to perform procedures using the steerable cannulas. In some examples, such systems can include a magnetically steerable screw-tip cannula and a torque applying mechanism. The torque applying mechanism can be located at a position toward the proximal end of the flexible cannula, outside the biological tissue. The torque applying mechanism can apply torque to the flexible cannula outside the biological tissue, and because the cannula has a high torsional stiffness the torque can be transferred to the screw-tip at the distal end of the cannula. The torque can cause the screw-tip to spin, which can generate insertion force or retraction force depending on the direction of spinning.

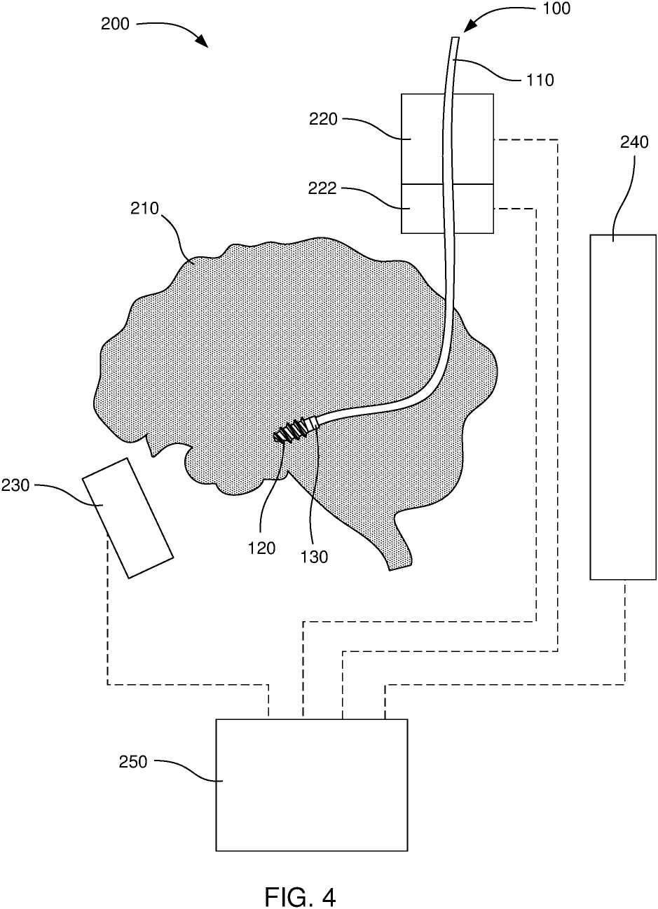

An example system 200 is shown in FIG. 4. This system includes a magnetically steerable screw-tip cannula 100 as described above. The cannula is fed into biological tissue 210 by a torque applying mechanism 220, which applies mechanical torque to the flexible cannula 110 at a location outside the biological tissue. The flexible cannula transfers the torque to a magnetically steerable screw-tip 120 to generate insertion or retraction force. A force sensor 222 is also included in this example. The force sensor can measure the mechanical torque applied to the flexible cannula. A magnetic field source 230 is positioned to generate a magnetic field impinging upon the magnet 130 of the magnetically steerable screw-tip cannula. The magnetic field can generate a force or torque on the screw-tip to cause deformation of the cannula and to steer the cannula. A location sensor 240 is positioned to locate the magnetically steerable screw-tip in the biological tissue. Locating the screw-tip in the tissue can be helpful for steering the screw-tip and avoiding certain sensitive portions of the tissue. The system also includes a controller 250. The controller is electrically connected to the torque applying mechanism, the force sensor, the location sensor, and the magnetic field source. These electrical connections are represented by dashed lines in the figure. The controller can be programmed to coordinate insertion or retraction of the cannula and magnetic steering via closed-loop control.

It is noted that the systems described herein can include various components to automate aspects of procedures performed using the steerable cannulas. For example, the torque applying mechanism can automate the application of torque to the cannula. However, this can also be done manually by a healthcare provider such as a surgeon manually rotating the proximal end of the cannula. Whether the torque is applied manually or by a mechanism, the torque can be applied at a location outside the biological tissue, i.e., outside the brain when the steerable cannula is inserted into the brain. Other aspects of procedures can also be performed manually or in an automated fashion by adding various components to the system. Several such components are described below.

The forces exerted by the flexible cannula on surrounding tissue can be smaller compared to steerable cannulas that are pushed into the tissue during insertion. When the insertion force is provided by pushing the cannula, the cannula can exert large stresses on the tissue in places where the cannula has curved due to steering. This can damage the tissue and cut through the tissue of the force is large enough. In contrast, the magnetically steerable screw-tip cannulas described herein do not derive the insertion force from pushing the cannula into the tissue. Rather, the insertion force is generated by the rotation of the screw-tip. The screw-tip moves forward during insertion because of the motion of the screw threads. Thus, the screw-tip pulls the flexible cannula behind it. Because the flexible cannula can be highly flexible (having a low bending stiffness), the flexible cannula does not exert a large force on the surrounding tissue as it is pulled behind the screw-tip.

In some examples, a smaller mechanical force, parallel to the lumen of the flexible cannula, can be applied to the flexible cannula from outside the tissue to help feed the flexible cannula into the tissue. However, the screw-tip can provide a majority of the insertion force for inserting the cannula into the tissue, and the additional force applied to the cannula from outside the tissue can be merely for the purpose of matching the speed of the screw-tip. Applying this additional mechanical force to the cannula from outside the tissue can help to further reduce stress placed on the tissue by the cannula as the cannula is inserted. Similarly, a mechanical retraction force can be applied to the cannula outside the tissue while the screw-tip is also rotated to generate a retraction force. The mechanical force applied to the cannula outside the tissue can be applied manually or by the torque applying mechanism described above.

The torque applying mechanism can include any suitable mechanism for applying mechanical torque to the flexible cannula. In various examples, the torque applying mechanism can include an electric motor, a pneumatic actuator, a piezoelectric actuator, or a combination thereof. In some examples, the flexible cannula can be fed through the torque applying mechanism as the torque is applied to the flexible cannula. In other examples, the torque applying mechanism can be fixed to the flexible cannula by clamping or another attachment method and the torque applying mechanism can move together with the cannula as the cannula is inserted or retracted. The torque applying mechanism can apply torque to the cannula in a direction parallel to the lumen of the cannula (i.e., the longitudinal axis of the cannula). The direction of a torque vector can be defined using the “right hand rule,” which states that the torque vector has a direction pointing perpendicular to the plane of rotation. This vector can be parallel to the lumen of the cannula. The direction of the vector can depend on the direction of rotation of the cannula.

The torque applying mechanism can also apply a force parallel to the lumen of the cannula, as explained above. In some examples, this force can have a magnitude from about 0.001 N to about 1 N. The system may also include a force sensor to measure the force and/or torque applied to the cannula. The force sensor can be integrated as a part of the torque applying mechanism or the force sensor can be a separate component.

The speed of insertion and retraction of the cannula can be controlled to be in a safe range for the particular procedure being performed. In some examples, the speed at which the screw-tip proceeds through the biological tissue during insertion can be from about 0.5 cm/minute to about 10 cm/minute, or from about 0.5 cm/minute to about 5 cm/minute, or from about 0.5 cm/minute to about 2 cm/minute, and in some cases up to 2 cm/sec. Similar speeds can be used when retracting the cannula from the biological tissue.

In more detail regarding the magnetic field source, in some examples the magnetic field source can include a permanent magnet. The permanent magnet can be oriented adjacent to the biological tissue and rotated and translated to affect the force and torque that are imparted on the magnet located at the screw-tip of the cannula. The pose of the permanent magnet can be controlled by a robot or manually. Alternatively, the magnetic field source can be an electromagnet. The electromagnet can be rotated and translated to affect the force and torque that are imparted on the magnet at the screw-tip of the cannula. Further, the electromagnet can be varied in magnetic field strength as an additional variable which can be used to actively control movement of the magnet at the screw-tip. For example, the electromagnet can have at least one current that is controlled to affect the force and torque that are imparted on the magnet at the screw-tip of the cannula. As with the permanent magnet, the electromagnet can be controlled by a robot or manually. In certain examples, the magnetic field source can include an omnidirectional magnet such as an Omnimagnet system, OctoMag system, or a system as described in U.S. Pat. No. 10,199,147. In another example, the magnetic field source can be a tri-axial Helmholtz coil. In certain examples, the magnetic field source can include multiple sets of wire coils oriented in different directions. The electric current through the wire coils can be controlled to generate magnetic fields oriented in different directions. The overall effective magnetic field of this magnetic field source can depend on the combination of the magnetic fields generated by the coils. Thus, an overall magnetic field can be generated with various orientations and magnetic field strengths.

The magnetic field strength generated by the magnetic field source can be from 0.1 mT to 100 mT at the location of the screw-tip of the cannula, and in some cases up to 8 T or higher, e.g. when using an MRI system as the magnetic field source. The magnetic field can be used to attract the magnets in the screw-tip, repel the magnets in the screw tip, to apply a torque to the magnets in the screw tip, or a combination of these. However, it is noted that in some examples the magnetic field source is not used to rotate the screw-tip around its longitudinal axis. The torque used to rotate the screw-tip around its longitudinal axis comes from the mechanical torque applied to the flexible cannula outside the biological tissue. Any torque applied to the screw-tip by the magnetic field source can be for the purpose of steering the screw tip.

A location sensor can be included in the system to locate the screw-tip within the biological tissue. In some examples, the location sensor can be a magnetic sensor that can provide orientation and location information about the screw-tip based on the magnets present in the screw-tip. In other examples, the location sensor can include a medical imaging device such as an X-ray device, a fluoroscopy device, a tomography device, an ultrasound sonography device, or a magnetic resonance imaging device. A magnetic resonance imaging device can be used if the magnet in the screw-tip is an electromagnet that may be turned off before the magnetic resonance imaging device is used. Any of these sensors can locate the screw-tip within the biological tissue. The sensor can be used periodically to locate the screw-tip, or the sensor can be turned on continuously to provide a real-time location of the screw-tip.

The system can also include a controller that can be electronically connected to some or all of the electronic components in the system. The controller can coordinate insertion of the cannula, retraction of the cannula, magnetic steering of the screw-tip, or a combination thereof. In some examples, the controller can be electronically connected to the torque applying mechanism, the force sensor, the location sensor, the magnetic field source, or a combination thereof. The controller can utilize closed-loop control, for example by signals to the torque applying mechanism to apply torque to the cannula, and adjusting the signals based on readings from the force sensor. In another example, the controller can adjust the placement, direction, and electric current to the magnetic field source based on readings from the location sensor. The controller can be programmed to insert the cannula along a desired pathway into biological tissue. The controller can also include a user interface to allow a user, such as a surgeon, to directly control the direction and speed of the screw-tip as the screw-tip cannula is inserted or retracted.

The present disclosure also describes methods of using magnetically steerable screw-tip cannulas. In some examples, a magnetically steered screw-tip cannula device can be inserted into a biological tissue such as a brain or other organ. The device can include a flexible cannula with a screw-tip having a magnet. The flexible cannula can be deformable by forces that can be safely imparted by biological tissue. The screw-tip can be located at a distal tip of the cannula, with an internal lumen that is substantially parallel with the screw axis and with the lumen of the cannula. The magnet can also be located near the distal tip of the cannula. The magnet can also have an internal lumen that is substantially parallel with the lumen of the cannula. The average magnetization of the magnet can be substantially parallel to the lumen. The lumens of the cannula, magnet, and screw-tip can form a continuous lumen. Mechanical torque can be applied parallel to the lumen of the cannula, at a position on the cannula outside of the biological tissue, to cause the cannula and its screw tip to rotate to generate insertion or retraction motion due to the screw. A magnetic field source can be disposed adjacent to the biological tissue so that a magnetic field produced by the magnetic field source impinges upon the magnet at the screw-tip of the cannula, leading to force and/or torque that cause deformation of the flexible cannula to steer the cannula through the tissue.

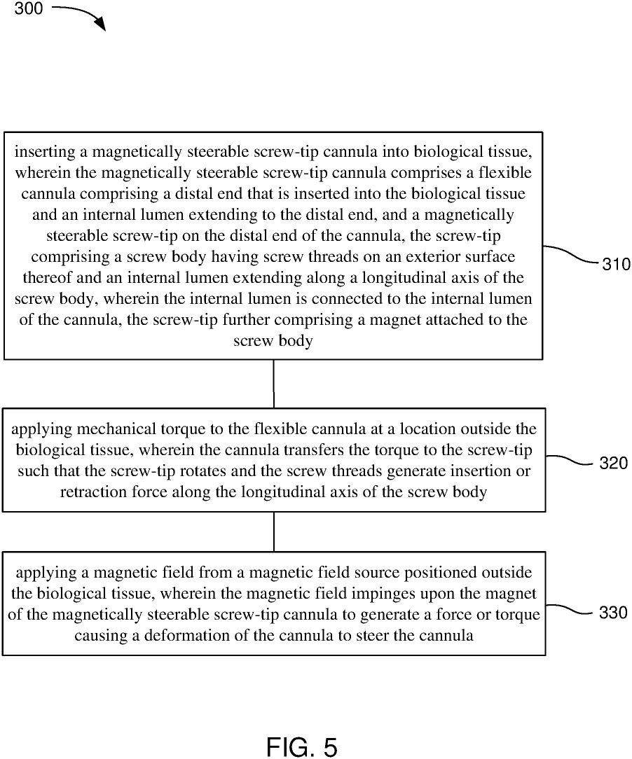

FIG. 5 is a flowchart illustrating and example method 300 of using a magnetically steerable screw-tip cannula. The method includes: inserting a magnetically steerable screw-tip cannula into biological tissue, wherein the magnetically steerable screw-tip cannula comprises a flexible cannula comprising a distal end that is inserted into the biological tissue and an internal lumen extending to the distal end, and a magnetically steerable screw-tip on the distal end of the cannula, the screw-tip comprising a screw body having screw threads on an exterior surface thereof and an internal lumen extending along a longitudinal axis of the screw body, wherein the internal lumen is connected to the internal lumen of the cannula, the screw-tip further comprising a magnet attached to the screw body 310; applying mechanical torque to the flexible cannula at a location outside the biological tissue, wherein the cannula transfers the torque to the screw-tip such that the screw-tip rotates and the screw threads generate insertion or retraction force along the longitudinal axis of the screw body 320; and applying a magnetic field from a magnetic field source positioned outside the biological tissue, wherein the magnetic field impinges upon the magnet of the magnetically steerable screw-tip cannula to generate a force or torque causing a deformation of the cannula to steer the cannula 330.

Methods of using magnetically steerable screw-tip cannulas can include using any of the components of the cannula devices and systems described above. In a particular example, a method of using a magnetically steerable screw-tip cannula can include controlling an electric current to an electromagnet that acts as a magnetic field source for steering the screw-tip cannula. Controlling the electric current can allow the strength of the magnetic field to be adjusted, which can provide finer control over magnetic steering of the screw-tip. Additionally, as mentioned above, some types of magnets include multiple coils that generate electric fields in different directions. The electric current through multiple coils can be controlled independent to allow the strength and direction of the overall magnetic field to be adjusted. In a particular example, the magnetic field source can include three wire coils that generate electric fields oriented in three mutually orthogonal directions. By independently controlling the electric current passing through these three coils, an overall magnetic field can be generated with any desired direction and strength.

In other examples, the magnetic field source can include a permanent magnet. Whether the magnetic field source is a permanent magnet or an electromagnet, it can be useful to move the magnetic field source in relation to the screw-tip to change the magnetic field strength and direction applied to the screw-tip. The movement of the magnetic field source can include translational movements, rotational movements, and combinations thereof. The methods described herein can include translating, rotating, or both translating and rotating the magnetic field source in order to affect the force and/or torque that is imparted to the magnet of the screw-tip cannula. The rotation and translation can be performed manually or by a robot. Examples of robot systems that can be useful for moving the magnetic field source include robotic arms and any other system with sufficient degrees of freedom to translate and rotate the magnetic field source with respect to the screw-tip.

The methods described above can also include locating the screw-tip using a location sensor. In one example, the screw-tip can be located at discrete time intervals and then the screw-tip can be moved by insertion, retraction, magnetic steering, or a combination thereof, between the times of locating. In another example, the location of the screw-tip can be continuously monitored while the screw-tip is inserted, retracted, magnetically steered, or a combination thereof. The screw-tip can be located using any of the location sensors described above.

The magnetically steerable screw-tip cannulas can be particularly useful for insertion into the brain to allow access to deep portions of brain while avoiding sensitive portions of the brain. The cannulas can also be useful for procedures involving other biological tissues, such as the lungs, spinal column, eyes, liver, pancreas, kidney, prostate, gastrointestinal tract, urinary tract, and others. The cannula devices and systems described above can all be used for any of these tissues.

Methods can also include using the cannula after the cannula has been inserted to a desired location in the biological tissue. The lumen of the cannula can be useful for delivering materials such as fluids to the tissue, or draining fluids from the tissue, or as a working channel for instruments, or to biopsy the tissue. In certain examples, a treatment fluid can be delivered through the cannula. Certain treatments can be useful to treat a specific area of the tissue, such as a tumor, infected area, and so on. However, it may be harmful to deliver the treatment, such as chemotherapy, radiation, antibiotics, etc., to healthy tissue. The magnetically steerable cannulas described herein can be useful for delivering such treatments directly to the affected areas of the tissue while reducing delivery to healthy tissue. In some cases, the internal lumen can be selectively blocked (e.g. blocked during insertion and then unblocked post insertion). This can prevent entry of undesired fluids or debris during insertion, for example. In additional alternatives, the internal lumen can be filled with an instrument. For example, ablation tools, sensing instruments, electrical stimulation instruments, electrical monitoring instruments, and others, can be permanently oriented within the internal lumen.

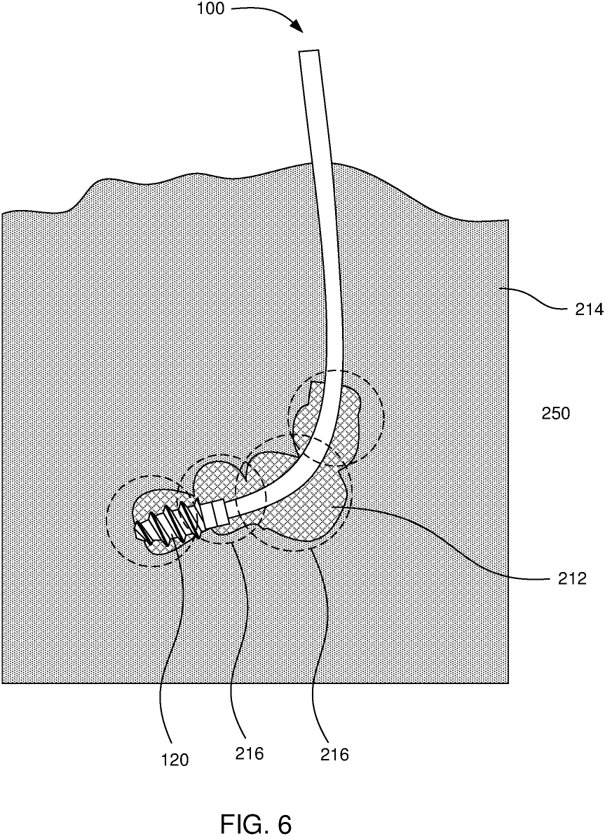

FIG. 6 shows an example of using a magnetically steerable screw-tip cannula 100 to deliver a treatment to a tumor 212 in a brain 214. The treatment can be a fluid delivered through the tip of the cannula. As the screw-tip 120 advances through the brain, the screw-tip can be steered to pass through the tumor. In this example, the tumor has an irregular shape. The treatment can be delivered to multiple treatment volumes 216 along the path of the cannula, either as the cannula is being inserted or as the cannula is being retracted. These roughly spherical treatment volumes form a “snowman” volume that closely matches the irregular shape of the tumor. Forming such a treatment volume would be difficult or impossible with a straight cannula. Thus, the steerable cannula described herein can allow for a safer treatment with less exposure of healthy brain tissue to the treatment fluid.

The flexible cannula can be designed to have a low bending stiffness so that the cannula can flex with a small radius of curvature. Smaller radii of curvature can allow the cannula to be steered to match irregular treatment volumes or to precisely avoid sensitive tissues. In some examples, the magnetically steerable screw-tip cannulas described herein can be steered with a minimum radius of curvature from 5 mm to 50 mm, or from 5 mm to 30 mm, or from 5 mm to 20 mm.

### EXAMPLES

A prototype magnetically steerable screw-tip cannula was constructed having a design similar to the design shown in FIGS. 1A-1B. The device was steered through an Agar gel-based tissue phantom and an ex vivo ovine brain. The prototype was composed of a hollow, 3-mm-long brass screw, connected to four 0.5-mm-long round, hollow ring magnets stacked, pulling a hollow PTFE tube. The insertion in the gel phantom was performed using an electric, mechatronic device which inserted and rotated the tube to drive the cannula forward, and a robotic device holding an external permanent magnet applying a magnetic torque to steer the cannula in an arc. Control of the cannula in the gel was performed with a human aligning the external permanent magnet via a software interface and advancing the screw via a software interface controlling the electric mechatronic system. The insertion in the ex vivo ovine brain was performed with manual insertion and rotation, i.e., a human held the tube near the insertion point and rotated it with their fingers. Steering was performed in the ovine brain with the same robotically controlled external permanent magnet.

Control of the external magnet was performed automatically using an open loop model of the cannula’s kinematics, i.e., the expected turning radius observed in gel.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example and not limitation, communication media includes wired media such as a wired network or direct-wired connection and wireless media such as acoustic, radio frequency, infrared and other wireless media. The term computer readable media as used herein includes communication media.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

What is claimed is:

1. A magnetically steerable screw-tip cannula, comprising:
a flexible cannula comprising a distal end to be inserted into biological tissue and an internal lumen extending to the distal end; and
a magnetically steerable screw-tip on the distal end of the cannula, the screw-tip comprising:
a screw body comprising screw threads on an exterior surface thereof, the screw threads oriented to generate insertion or retraction force along a longitudinal axis of the screw body when the screw body rotates, the screw body further comprising an internal lumen extending along the longitudinal axis of the screw body, wherein the internal lumen is connected to the internal lumen of the cannula, and
a magnet associated with the screw body having an average magnetization substantially parallel to the longitudinal axis of the screw body.

2. The magnetically steerable screw-tip cannula of claim 1, wherein the screw body is made of a metal, a polymer, or a combination thereof.

3. The magnetically steerable screw-tip cannula of claim 1, wherein the magnets are attached to the screw body.

4. The magnetically steerable screw-tip cannula of claim 1, wherein the screw body is formed of the magnets.

5. The magnetically steerable screw-tip cannula of claim 1, wherein the magnet is a permanent magnet.

6. The magnetically steerable screw-tip cannula of claim 5, wherein the permanent magnet comprises two or more stacked permanent magnets.

7. The magnetically steerable screw-tip cannula of claim 1, wherein the magnet comprises a soft-magnetic material that becomes magnetized in the presence of an applied magnetic field.

8. The magnetically steerable screw-tip cannula of claim 1, wherein the magnet is an electromagnet.

9. The magnetically steerable screw-tip cannula of claim 1, wherein the magnet has an internal lumen that is substantially coaxial with the internal lumen of the screw body.

10. The magnetically steerable screw-tip cannula of claim 1, wherein the magnet is shaped as a cylinder or ring.

11. The magnetically steerable screw-tip cannula of claim 1, further comprising one or more magnets distributed along a length of the flexible cannula.

12. The magnetically steerable screw-tip cannula of claim 1, wherein the flexible cannula has a controlled bending stiffness due to at least one of selective etched patterning through walls of the flexible cannula and selective removal of material.

13. The magnetically steerable screw-tip cannula of claim 1, wherein the internal lumen includes at least one of: a selective block adapted to allow blocking of the internal lumen during insertion and an instrument permanently oriented within the internal lumen.

14. A system comprising the magnetically steerable screw-tip cannula of claim 1 and a torque applying mechanism at a position toward a proximal end of the flexible cannula, the torque applying mechanism being configured to apply torque to the flexible cannula such that the flexible cannula transfers the torque to the magnetically steerable screw-tip to generate insertion or retraction force.

15. The system of claim 14, wherein the torque applying mechanism applies the torque using an electric motor, pneumatic actuator, piezoelectric actuator, or combination thereof.

16. The system of claim 14, wherein the torque applying mechanism also applies a mechanical force parallel to the lumen of the cannula to assist in insertion or retraction.

17. The system of claim 14, further comprising a magnetic field source positioned to generate a magnetic field impinging upon the magnet of the magnetically steerable screw-tip cannula to generate a force or torque causing a deformation of the cannula to steer the cannula.

18. The system of claim 17, wherein the magnetic field source comprises a permanent magnet or an electromagnet that impart force or torque on the magnet of the magnetically steerable screw-tip cannula.

19. The system of claim 18, wherein the magnetic field source further comprises a robot to rotate the permanent magnet or the electromagnet, to translate the permanent magnet or the electromagnet, to control an electric current to the electromagnet, or a combination thereof, in order to affect the force or torque that is imparted to the magnet of the magnetically steerable screw-tip cannula.

20. The system of claim 14, further comprising a location sensor positioned to locate the magnetically steerable screw-tip in the biological tissue, wherein the location sensor comprises a magnetic sensor, a medical imaging device, an X-ray device, a fluoroscopy device, a tomography device, an ultrasound device, a magnetic resonance imaging device, or a combination thereof.

21. The system of claim 20, further comprising a force sensor to measure mechanical torque applied to the cannula, mechanical force applied to the cannula, or a combination thereof.

22. The system of claim 21, further comprising a controller electronically connected to at least one of the location sensor and the force sensor, wherein the controller coordinates insertion or retraction and magnetic steering via closed-loop control.

23. A method of using a magnetically steerable screw-tip cannula, comprising:
inserting a magnetically steerable screw-tip cannula into biological tissue, wherein the magnetically steerable screw-tip cannula comprises:
a flexible cannula comprising a distal end that is inserted into the biological tissue and an internal lumen extending to the distal end, and
a magnetically steerable screw-tip on the distal end of the cannula, the screw-tip comprising a screw body having screw threads on an exterior surface thereof and an internal lumen extending along a longitudinal axis of the screw body, wherein the internal lumen is connected to the internal lumen of the cannula, the screw-tip further comprising a magnet associated with the screw body;
applying mechanical torque to the flexible cannula at a location outside the biological tissue, wherein the cannula transfers the torque to the screw-tip such that the screw-tip rotates and the screw threads generate insertion or retraction force along the longitudinal axis of the screw body; and
applying a magnetic field from a magnetic field source positioned outside the biological tissue, wherein the magnetic field impinges upon the magnet of the magnetically steerable screw-tip cannula to generate a force or torque causing a deformation of the cannula to steer the cannula.

24. The method of claim 23, wherein the magnetic field source is an electromagnet having at least one current that is controlled to affect the force or torque that are imparted on the magnet located near the distal tip of the cannula.

25. The method of claim 23, further comprising using the internal lumen to deliver fluids to the biological tissue, to drain fluids from the biological tissue, as a working channel for instruments, selectively blocking the internal lumen during insertion, or a combination thereof.

26. The method of claim 23, wherein the biological tissue is a brain, lung, spinal column, eye, liver, pancreas, kidney, prostate, gastrointestinal tract, urinary tract, muscle, adipose, or a combination thereof.

27. The method of claim 23, wherein the mechanical torque is applied manually or using a torque applying mechanism.

28. The method of claim 23, wherein the magnetic field source comprises a permanent magnet or electromagnet and wherein the method comprises rotating the permanent magnet or electromagnet, translating the permanent magnet or electromagnet, controlling an electric current to the electromagnet, or a combination thereof, in order to affect the force or torque that is imparted to the magnet of the magnetically steerable screw-tip cannula.

29. The method of claim 28, wherein the rotation, translation, control of the electric current, or a combination thereof are performed manually or by a robot.

30. The method of claim 23, further comprising locating the magnetically steerable screw-tip in the biological tissue using a magnetic sensor, a medical imaging device, an X-ray device, a fluoroscopy device, a tomography device, an ultrasound device, a magnetic resonance imaging device, or a combination thereof.
